(19) United States
(12) Patent Application Publication
(10) Pub. No.: US 2007/0202091 A1
(43) Pub. Date: Aug. 30, 2007

(54) Nutraceutical composition and method for promoting the rejuvenation of cells in warm-blooded animals including humans

(76) Inventor: Stephen L. DeFelice, Westfield, NJ (US)

Correspondence Address: Allen R. Kipnes, Esq.;WATOV & KIPNES, P.C., P.O. Box 247, Princeton Junction, NJ, 08550, US

(21) Appl. No.: 11/711,588

(22) Filed: Feb. 27, 2007

Related U.S. Application Data
(60) Provisional application No. 60/777,471, filed on Feb. 28, 2006.

(51) Int. Cl. A61K 38/43; A61K 31/045; A61K 31/14; A61K 31/341; A61K 31/56; A61K 33/04; A61K 33/06

(52) U.S. Cl. 424/94.1; 424/682; 424/702; 514/169; 514/2; 514/440; 514/474; 514/642; 514/675; 514/724; 514/12; 514/171; 424/439

(57) ABSTRACT

A nutraceutical composition and method for promoting the rejuvenation of cells in warm-blooded animals including humans employing mitochondrial energy production-inducing agent for inducing the mitochondrial energy production to proceed from a lower energy production state to a higher energy production state and a nutraceutical composition for supporting the metabolic requirements of the increased mitochondrial energy production induced by the mitochondrial energy production-inducing agent.

### FIELD OF THE INVENTION

The present invention relates to nutraceutical compositions and methods for promoting the rejuvenation of cells in warm-blooded animals including humans using the same.

### BACKGROUND OF THE INVENTION

Every cell in the body contains tiny organelles referred to as “mitochondria.” Mitochondria are intracellular powerhouses that produce most of the energy used by the body. Their primary function is to convert organic materials such as carbohydrates and fat into energy in the form of adenosine triphosphate (ATP) via the process of oxidative phosphorylation. ATP is the body's principal source of cellular energy. Such mitochondrial energy production is necessary for physical strength, energy, stamina and life itself. A small decrease in mitochondrial energy output can result in weakness, fatigue, cognitive difficulties, and inefficiency in repairing damage to macromolecules, cells, tissues and organs leading to deterioration and breakdown.

The total output of mitochondrial energy production declines with age. It is believed that free radicals and oxidants cause adverse changes in mitochondria including damage to mitochondrial DNA, decreased efficiency, altered response and decreased membrane fluidity. Research has shown that many common age-related diseases and conditions including, but not limited to, Parkinsons disease, Alzheimers disease, heart disease, fatigue syndromes, muscle weakness, osteoporosis, and other conditions associated with ageing, are related to a reduction in mitochondrial energy production. For example, muscle cells and mitochondria are markedly diminished in number and size, which diminishes total energy output.

All living organisms undergo a process of cellular deterioration and declining efficiency resulting from a reduction in ATP production, leading to degeneration, damage, ageing, breakdown and death. This process is further characterized by a decline in the ability to respond to stress, an increase in homeostatic imbalance and an increase in the risk of disease, all of which is associated with ageing or senescence. Thus, mitochondrial energy production is closely tied to the efficiency of DNA repair, production of antioxidant enzymes, rates of free radical production and other factors that influence ageing.

Nutrition plays a critical role in maintaining good health. Proper nutrition prevents or reduces the rate of dietary deficiencies, and also protects against the development of disease. Proper nutrition plays an increasingly important role as the body faces physiological stress including stress associated with ageing.

Many nutrients have an indispensable role in mitochondrial energy production and provide vital antioxidant protection against deleterious free radicals generated by oxidative phosphorylation in the mitochondria. Such nutrients which have a positive effect on mitochondrial energy production include, but are not limited to, vitamin B1, vitamin B2, vitamin B3, vitamin C, vitamin E, vitamin K, ethylenediaminetetraacetic acid, coenzyme Q10, idebenone, succinates, dichloroacetate, acetyl-L-carnitine, L-carnitine, alpha phenyl t-butyl nitrone, N-acetyl-cysteine, alpha-lipoic acid, ginkgo biloba, D-ribose, creatine, taurine, and omega-3 fatty acids. Sufficient amounts of such nutrients are needed to ensure efficient mitochondrial energy production, and the availability of such nutrients has been shown to influence the progression of diseases and conditions related to ageing.

Therefore it is widely believed that administering anti-oxidants and nutrients in ample amounts can positively affect mitochondrial energy production, and slow down the effects of ageing. However, these nutrients are not effectively utilized when the mitochondrial energy production, or ATP generating capacity, of the individual has declined substantially. As mitochondrial energy production in cells decline over time, the ability of such cells to utilize such nutrients is likewise greatly diminished. Thus, once ageing becomes pronounced, the cells can no longer use the desired nutrients to elevate mitochondrial energy production. Since mitochondrial energy levels cannot be elevated there is a further decline and deterioration associated with ageing. Therefore the use of nutrients, which have a positive effect on mitochondrial energy production alone to slow down or reverse the ageing process, had limited success.

Accordingly, there is a need to address the ageing process and to slow down or even reverse the effects of ageing. There is a further need to provide compositions and methods of use, which can induce elevated levels of mitochondrial energy production in cells, and supply necessary nutrients to meet and sustain the elevated levels of mitochondrial energy production in order to provide anti-ageing benefits.

### SUMMARY OF THE INVENTION

The present invention is related to a nutraceutical composition and method of using the same for promoting the rejuvenation of cells in a warm-blooded animal including a human. The present invention provides a method wherein mitochondrial energy production is induced from a lower energy production state to a higher energy production state in the body of the warm-blooded animal. In accordance with the present invention, the cells in the warm-blooded animals are stimulated to increase mitochondrial energy production (e.g., adenosine triphosphate production), which creates an energy nutrient deficient state, while minimizing oxidant activity. As part of the present invention, there is provided a unique balanced composition formulated to provide the nutrients necessary to sustain the increased mitochondrial energy production essential for providing anti-ageing benefits.

In one aspect of the present invention, there is provided a method for rejuvenating cells having a lower energy production state to a higher energy production state in a warm-blooded animal including a human, the method comprising the steps of:

inducing the lower energy production state cells to increase mitochondrial energy production toward the higher energy production state; and

supplying a nutraceutical composition for uptake by the higher energy production state cells in amounts sufficient to satisfy the metabolic requirements resulting from the increase in mitochondrial energy production.

In another aspect of the present invention, there is provided a nutraceutical composition for administration to a patient having received a mitochondrial energy production-inducing agent to elevate the energy production of the mitochondria from a lower energy production state to a higher energy production state, wherein the existence of the higher energy production state results in a deficiency of energy nutrients necessary to sustain the higher energy production state. The nutraceutical composition comprises at least one energy nutrient in an amount effective for supplying the nutrients needed to sustain the higher energy production state, and a nutraceutically acceptable carrier.

### DETAILED DESCRIPTION OF THE INVENTION

The present invention is directed to a nutraceutical composition and method for promoting the rejuvenation of cells in a warm-blooded animal including a human. The present invention provides a method wherein mitochondrial energy production is induced to increase from a lower energy production state to a higher energy production state in the body of the warm-blooded animal. In accordance with the present invention, the cells in the warm-blooded animals are stimulated to increase mitochondrial energy production (e.g., adenosine triphosphate production), which creates an energy nutrient deficient state, while minimizing oxidant activity. The stimulated cells are able to more efficiently utilize the nutrients administered to the warm-blooded animal to satisfy the demand, thereby reversing, halting or slowing down cellular deterioration. As part of the present invention, there is provided a unique balanced composition of one or more energy nutrients formulated to meet the need and thereby sustain the desired mitochondrial energy production essential for providing anti-ageing benefits. The method of the present invention provides means for at least slowing down or reversing the progress of ageing.

As used herein, the term “mitochondrial energy production” shall mean the energy produced by cells within the body of warm-blooded animals, including humans, typically in the form of adenosine triphosphate (ATP). The energy is produced in a reaction in which organic materials such as carbohydrates and fats are converted to adenosine triphosphate (ATP) via oxidative phosphorylation.

As used herein, the term “lower energy production state” shall mean a level of mitochondrial energy production routinely observed in ageing individuals including those of the elderly or geriatric age groups. The term “higher energy production state” shall mean a level of mitochondrial energy production higher than the level of the lower energy production state, and preferably, shall mean a level of mitochondrial energy production routinely observed in growing and maturing individuals including those of the adolescent to young adult age groups.

The term “nutraceutical composition” shall mean the particular components and amounts of the components in the form of energy nutrients, which are employed in the present invention to meet the nutrient deficiency generated by the stimulation of the cells and the resultant increase in mitochondrial energy production (e.g., ATP production). The term “energy nutrients” shall be used to describe in broad terms the materials, which may be fat- or water-soluble, needed to satisfy the demand by cells once they have been stimulated to increase mitochondrial energy production from a lower energy production state to a higher energy production state.

The mitochondria of cells in warm-blooded animals including humans require energy nutrients for implementing optimal mitochondrial energy production. However, as celIs age, the mitochondrial energy production slows down over time, and the cell's capacity to take up and utilize the energy nutrients likewise diminishes. Such ageing cells exhibit a relatively lower energy production state, and generally do not respond to the energy nutrients because the reduced demand for energy nutrients when the cells are operating at the lower energy production state. Applicant believes that when the cells are induced to enter a higher energy production state, the demand for nutrients increases, and the ageing cells will thereby rapidly absorb and utilize the elevated levels of energy nutrients for energy production. In this manner, the anti-ageing benefits are therefore achieved by the sustained upgrade in energy production.

This energy nutrient deficient state can be achieved by stimulating cells to increase mitochondrial energy production from a lower energy production state to a higher energy production state, which creates an increased metabolic demand and utilization of the energy nutrients. The increased mitochondrial energy production is generally exhibited by an increase in adenosine triphosphate (ATP) production.

Accordingly, the present invention provides a nutraceutical composition and method for promoting the rejuvenation of cells having a lower energy production state to a higher energy production state in a warm-blooded animal including a human. The method generally comprises inducing the lower energy production state cells to increase mitochondrial energy production toward the higher energy production state, and supplying a nutraceutical composition for uptake by the higher energy production state cells in amounts sufficient to satisfy the metabolic requirements resulting from the increase in mitochondrial energy production.

The mitochondrial energy production is increased through administration of a mitochondrial energy-inducing agent in sufficient amounts to proceed from a lower energy production state to a higher energy production state. The mitochondrial energy-inducing agent comprises any substance including hormones capable of positively elevating or enhancing the rate of mitochondrial energy production in warm-blooded animals including humans. Preferred examples of the mitochondrial energy-inducing agent may be selected from growth hormone (GH), a steroid, and mixtures thereof. More preferably, the growth hormone (GH) is recombinant human growth hormone (rhGH), and the steroid is testosterone.

As used herein, the terms “sufficient amount” or “effective amount” mean amounts of the mitochondrial energy production-inducing agent for use in the methods of the present invention, which are capable of increasing the mitochondrial energy production of adenosine triphosphate to the higher energy production state. The specific dose of a mitochondrial energy production-inducing agent administered according to the present invention will, of course, be determined by the particular circumstances of the patient including, for example, the compound administered, the route of administration, the general health of the patient, body weight, and the severity of the mitochondrial ATP output deficiency being treated, and may be readily determined by one skilled in the art..

A typical daily dose will contain a non-toxic dosage level of from about 0.01 pg/kg/day to about 1000 mg/kg/day of a mitochondrial energy production-inducing agent of the present invention. Preferred daily doses generally will be from about 1 μg/kg/day to about 200 mg/kg/day. The dose of the steroid will typically be toward the upper end of the range while the amount of growth hormone will typically be at the lower end of the range. During the course of the treatment, the concentration of the mitochondrial energy production-inducing agent present in the body may be monitored to ensure that the desired level of mitochondrial energy production is maintained.

For example, testosterone may be formulated for administration to a patient at a daily dose of about 100 mg. The therapeutic goal is to maintain a total testosterone level of at least 500 ng/dL. If serum testosterone concentration is greater than 1000 ng/dL, the dose is decreased from 100 mg to 75 mg daily. If testosterone concentration is less than 500 ng/dL, the dose is increased from 100 mg to 125 mg daily.

In another example of the present invention, the dosage amount of recombinant human growth hormone may be about 5 μg/kg/day. In order to acclimate the patient to rhGH and reduce the potential for adverse events, the patient is to receive about 2.5 pg/kg/day, for about the first 28 days before increasing the dose to about 5 μg/kg/day. The dosage amounts of the mitochondrial energy production inducing-agent utilized will depend on several factors including, but not limited to, the agent administered, the duration of the treatment regimen, the patient's age, gender, health, tolerance, and energy nutrient needs or deficiency, the level of mitochondrial energy production, toxicity, and the like, and may be readily determined by one skilled in the art.

The mitochondrial energy production-inducing agent of the present invention may be administered by a variety of routes including oral, topical, parenteral, rectal, transdermal, subcutaneous, intravenous, intramuscular, and intranasal. The mitochondrial energy production-inducing agents are preferably formulated prior to administration, the selection of which will be decided by the attending physician. Typically, a mitochondrial energy production-inducing agent is formulated with a suitable pharmaceutically acceptable carrier, diluent or excipient to form a pharmaceutical composition.

The total active ingredients in such compositions comprises from 0.1% to 99.9% by weight of the composition. The term “pharmaceutically acceptable” shall mean that the carrier, diluent, excipients, and/or salt must be compatible with the other ingredients of the composition, and not deleterious to the recipient thereof.

Pharmaceutical compositions containing a mitochondrial energy production-inducing agent can be prepared by procedures known in the art using well known and readily available ingredients. For example, the mitochondrial energy production-inducing agent can be formulated with common excipients, diluents, or carriers, and formed into tablets, capsules, suspensions, powders, and the like. Examples of excipients, diluents, and carriers that are suitable for such compositions include the following fillers and extenders such as starch, sugars, mannitol, and silica derivatives binding agents such as carboxymethyl cellulose and other cellulose derivatives, alginates, gelatin, and polyvinyl-pyrrolidone, moisturizing agents such as glycerol, disintegrating agents such as calcium carbonate and sodium bicarbonate agents for retarding dissolution such as paraffin resorption accelerators such as quaternary ammonium compounds, surface active agents such as cetyl alcohol, glycerol monostearate, adsorptive carriers such as kaolin and bentonite, and lubricants such as talc, calcium and magnesium stearate, and solid polyethylene glycols.

The mitochondrial energy production-inducing agents can also be formulated as elixirs or solutions for convenient oral administration or as solutions appropriate for parenteral administration, for example, by intramuscular, subcutaneous or intravenous routes.

Additionally, the mitochondrial energy production-inducing agent may be well suited for compositions suitable for sustained release administrations. The compositions can be so constituted that they release the active ingredient only or preferably in a particular physiological location, possibly over a period of time. The coatings, envelopes, and protective matrices may be made, for example, from polymeric substances or waxes.

In a preferred embodiment of the present invention, the method comprises stimulating the cells to produce increased levels of adenosine triphosphate, and supplying a nutraceutical composition for uptake by the cells in amounts sufficient to satisfy the metabolic requirements by addressing the deficiency of nutrients resulting from the increased production levels of adenosine triphosphate in the cells. The stimulation of the cells is achieved by administering a sufficient amount of the mitochondrial energy production-inducing agent to support the production of increased levels of adenosine triphosphate production in the cells.

In a more preferred embodiment of the present invention, the method further comprises measuring the progress of cell rejuvenation attained, and adjusting the mitochondrial energy production for optimizing the cell rejuvenation progress in slowing or reversing the effects of ageing. The progress of cell rejuvenation may be measured by examining physical and mental performance of the individual receiving treatment, including, but not limited to, muscle strength and tone, body mass index, stamina, agility, flexibility, balance, cardiovascular health, neurological health, sensory perception, mood, memory, cognitive abilities, and the like. The mitochondrial energy production levels may be adjusted by fine-tuning the administration of the mitochondrial energy production-inducing agent and/or the formulation of the energy nutrients in the nutraceutical composition to the warm-blooded animal.

In another embodiment of the present invention, there is provided a nutraceutical composition for administration to a patient having received a mitochondrial energy production-inducing agent to elevate the energy production of the mitochondria from a lower energy production state to a higher energy production state, wherein the existence of the higher energy production state results in a deficiency of energy nutrients necessary to sustain the higher energy production state. The nutraceutical composition comprises at least one energy nutrient in an amount effective for supplying the nutrients needed to sustain the higher energy production state, and a nutraceutically acceptable carrier.

The nutraceutical composition generally comprises one or more energy nutrients selected from fat-soluble energy nutrients, water-soluble energy nutrients or mixtures thereof. These nutrients are selected because of their individual and synergistic capacity to meet the deficiency of energy nutrients in the cells resulting from the enhanced ATP production that occurs once the cells have been stimulated. It is critical to provide sufficient amounts of such energy nutrients to the cells when the enhanced ATP production output is generated through the use of the mitochondrial energy production-inducing agent.

In general, water-soluble energy nutrients are more rapidly excreted from the body, while fat-soluble energy nutrients remain in the body for much longer periods of time. Therefore, increased amounts of water-soluble energy nutrients may be administered relative to fat-soluble energy nutrients over a given regimen period. Alternatively, the effective dosage amounts of at least one of the water-soluble energy nutrient and fat-soluble energy nutrient may be varied relative to the other over the period of time to avoid potential adverse effects associated with excess stores of the fat-soluble energy nutrient in the body of the warm-blooded animal including human. Such potentially adverse effects include diarrhea, abdominal cramps, bloating, and the like, that may be experienced especially in elderly patients. As a result, the nutraceutical composition is orally tolerable and more physiologically acceptable to the patient, while providing the sufficient amount of energy nutrients needed to sustain the increased mitochondrial energy production. This general regimen for administering energy nutrients substantially enhances patient compliance.

Nutraceutical compositions of the present invention include energy nutrients selected from amino acids, quaternary amines, vitamins, dietary minerals, chelating agents, enzymes, ubiquinones, antioxidants, essential fatty acids, carotenoids, tocopherols and mixtures thereof, which are generally underutilized in lower energy production state cells exhibiting low mitochondrial ATP production output (i.e., ageing cells or damaged cells). However, if such low output cells are stimulated, ATP production increases, a deficiency in the above-mentioned nutrients is created and the nutraceutical composition is used to overcome the deficiency and thereby meet and sustain elevated ATP production.

Preferably, the nutraceutical composition is composed of at least one of the following energy nutrients including, but not limited to, vitamin B1, vitamin B2, vitamin B3, vitamin C, at least one tocopherol, vitamin K (vitamin E), ethylenediaminetetraacetic acid, coenzyme Q10, idebenone, succinates, dichloroacetate, acetyl-L-carnitine, L-carntine, alpha phenyl t-butyl nitrone, N-acetyl-cysteine, alpha-lipoic acid, ginkgo biloba, D-ribose, creatine, taurine, omega-3 fatty acids, magnesium, selenium, and carotenoid.

More preferably, the nutraceutical composition is composed of a water-soluble portion selected from carnitine, magnesium, vitamin C, selenium and mixtures thereof, a fat-soluble portion selected from tocopherols, carotenoids, coenzyme Q10, alpha-lipoic acid, and mixtures thereof, wherein the amount of the fat-soluble portion is varied relative to the water-soluble portion on a day-to-day basis to minimize any adverse effects as described above.

In a particular preferred embodiment of the present invention, there is provided a nutraceutical composition comprising:
50 mg to 15,000 mg of carnitine, preferably 100-10,000 mg,
10 mg to 2,000 mg of magnesium, preferably 50-1,000 mg,
10 mg to 1,000 mg of vitamin C, preferably 50-600 mg,
5 ug to 500 ug of selenium, preferably 10-400 ug,
5 mg to 600 mg of coenzyme Q10, preferably 10-400 mg
5 mg to 500 mg of alpha-lipoic acid, preferably 10-250 mg,
5 mg to 800 mg of at least one tocopherol, preferably 10-500 mg,
1 mg to 600 mg of at least one carotenoid, preferably 3-500 mg

The nutraceutical compositions of the present invention may comprise or use a formulation of vitamins and minerals, in either chelated or non-chelated form, that work together with various metabolic systems and physiological responses of the human body. The energy nutrients are available from numerous commercial sources, and in several active forms or salts thereof, known to those of ordinary skill in the art. Hence, the nutraceutical compositions and methods of the present invention are not limited to comprising or using any particular form of the vitamin or mineral ingredient as described herein.

The nutraceutical compositions of the present invention are intended for administration to a warm-blooded animal including a human, in a suitable dosage form as is known in the art. Suitable dosage forms known in the art include parenteral, enteral, and especially oral. Oral solid and liquid dosage forms are particularly preferred.

Oral solid dosage forms are well known in the art and include tablets, caplets, gelcaps, capsules, and edible food items. Oral solid dosage forms can be made with one or more nutraceutically acceptable carriers. Nutraceutically acceptable carriers assist or make possible the formation of a dosage form for a bioactive material and include diluents, binding agents, lubricants, glidants, disintegrants, coloring agents, and flavorants and nutrients. A carrier is nutraceutically acceptable if, in addition to performing its desired function, it is non-toxic, well tolerated upon ingestion, and does not interfere with absorption of bioactive materials.

The ingredients of the present invention may be formulated into a nutraceutical composition which may be in the form of a solid powder, caplets, tablets, lozenges, pills, capsules, or a liquid, and which may be administered alone or in suitable formulation with other components. For example, the nutraceutical composition of the present invention may be administered in one or more caplets or lozenges as practical for ease of administration. Each of the vitamins and minerals is commercially available, and can be blended to form a single composition or can form multiple compositions, which may be co-administered. More preferably, the composition of the present invention is in the form of a powder form for subsequent reconstitution by addition of a liquid into a beverage for consumption by the patient.

To prepare the nutraceutical compositions of the present invention, each of the active ingredients may be formulated as intimate admixture with a suitable nutraceutically acceptable carrier according to conventional compounding techniques. The nutraceutically acceptable carrier may take a wide variety of forms depending upon the form of the preparation desired for administration, e.g., oral, sublingual, nasal, via topical patch, or parenteral.

In preparing the nutraceutical composition in a dosage form selected from oral, topical and parenteral, any usual media may be utilized. For liquid preparations (e.g., suspensions, elixirs, and solutions), media containing, for example water, oils, alcohols, flavoring agents, preservatives, coloring agents and the like may be used. Carriers such as starches, sugars, diluents, granulating agents, lubricants, binders, disintegrating agents and the like may be used to prepare oral solids (e.g., powders, caplets, pills, tablets, capsules, and lozenges). Controlled release forms may also be used. Because of their ease in administration, caplets, tablets, pills, and capsules represent the most advantageous oral dosage unit form, in which case solid carriers are employed. If desired, tablets may be sugar coated or enteric coated by standard techniques. All of these pharmaceutical carriers and compositions are well known to those of ordinary skill in the art. See generally, e.g., Wade & Waller, Handbook of Pharmaceutical Excipients (2nd ed. 1994).

The administration of the nutraceutical composition will typically be administered over several weeks in a manner, which provides an effective amount of each of the desired nutrients so as to ensure that the demand for the nutrients by the induced mitochondrial energy production or stimulated ATP production, is satisfied.

The nutraceutical composition may be formulated in a unit dose form. Such unit dose will generally comprise an amount of each energy nutrient in the range of from about 0.01 μg to about 15,000 mg, more usually about 0.1 μg to about 1000 mg, and more especially about 0.1 μg to about 500 mg. The energy nutrients may be taken in doses, one to six times a day in a manner such that the total daily dose for a 70 kg adult will generally be in the range of from about 0.1 μg to 40,000 mg, and more usually about 1 μg to 10,000 mg, generally from about 0.5 μg to 20,000 mg. The daily dose of the energy nutrients is usually about 0.1 μg to about 50 mg/kg body weight. Usually about 0.5 μg to about 40 mg/kg/day and preferably about 1.0 μg to about 20 mg/kg/day are effective in one or several administrations per day in order to obtain the desired results. Of course, the administered dose depends on the age, state of health, and weight of the recipient, the extent of the mitochondrial activity, the type of additional treatments that may be carried out at the same time, and the type of desired effect. During the course of the treatment, the concentration of the nutraceutical compositions may be monitored to ensure that the desired level is maintained.

The nutraceutical composition may be administered in the form of daily doses such as in liquid, tablet, capsule or pill form. Alternatively, the nutraceutical composition may be administered via one or more dosage forms each comprising varying amounts of at least one of water-soluble and fat-soluble energy nutrients over a period of time.

In one embodiment of the present invention, a first dosage form containing primarily water-soluble energy nutrients (e.g. carnitine, magnesium and selenium) followed by a second dosage form containing water soluble and fat-soluble energy nutrients. Coenzyme Q₁₀ may be included in either or both dosage forms. Preferably, the this mode of administration may be incorporated into a regimen implemented over a period of time to balance the respective retention and performance characteristics of the water-soluble and fat-soluble energy nutrients in the body, and thereby maintain sufficient amounts of both water- and fat-soluble energy nutrients in the body to sustain the increased mitochondrial energy production. Furthermore, this mode of administration wherein the effective dosage amounts of the water-soluble portion and the fat-soluble portion of the present nutraceutical composition is varied over the treatment period, at least minimizes or avoids adverse effects as described above.

The nutraceutical composition may be formulated to comprise ratio amounts of a water-soluble portion to fat-soluble portion in a range of from about 100 to 1 to 0.01 to 1 by weight based on the total weight of the nutraceutical composition excluding the carrier and excipients. Preferably, the daily amount of the water-soluble portion will be greater than the daily amount of the fat-soluble portion, because the fat-soluble portion will be retained in the body for a longer period of time relative the water-soluble portion. The ratio amounts of the water-soluble portion and fat-soluble portion utilized will depend on several factors including, but not limited to, the energy nutrients formulated, the duration of the treatment regimen, the patient's age, gender, health, oral tolerance, and energy nutrient needs or deficiency, the level of mitochondrial energy production, and the like.

In one example to exemplify the present invention, the nutraceutical composition is formulated for a seven-day regimen. For Day 1 dosage, the nutraceutical composition is formulated with about 4,050 mg of the water-soluble portion, and about 450 mg of the fat-soluble portion to yield an amount ratio of water-to fat-soluble portions of about 9 to 1. For Day 2 dosage form, the nutraceutical composition is formulated with about 4,050 mg of the water-soluble portion, and about 450 mg of the fat-soluble portion to yield an amount ratio of about 9 to 1. For Day 3 dosage form, the nutraceutical composition is formulated with about 3,850 mg of the water-soluble portion, and about 650 mg of the fat-soluble portion to yield an amount ratio of about 6 to 1. For Day 4 dosage form, the nutraceutical composition is formulated with about 2,800 mg of the water-soluble portion, and about 1,000 mg of the fat-soluble portion to yield an amount ratio of about 2.8 to 1. For Day 5 dosage form, the nutraceutical composition is formulated with about 2,700 mg of the water-soluble portion, and about 1,000 mg of the fat-soluble portion to yield an amount ratio of about 2.7 to 1. For Day 6 dosage form, the nutraceutical composition is formulated with about 2,500 mg of the water-soluble portion, and about 1,000 mg of the fat-soluble portion to yield an amount ratio of about 2.5 to 1. For Day 7 dosage form, the nutraceutical composition is formulated with about 2,700 mg of the water-soluble portion, and about 1,000 mg of the fat-soluble portion to yield an amount ratio of about 2.7 to 1.

### EXAMPLE 1

### Drug Dosage Regimen of Mitochondrial Energy Production-inducing Agents Comprising Testosterone and Growth Hormone

Testosterone formulated in a gel carrier is administered topically to a patient at a daily dose of about 100 mg. The therapeutic goal is to maintain a total testosterone level of at least 500 ng/dL. If serum testosterone concentration is greater than 1000 ng/dL, the dose is decreased from 100 mg to 75 mg daily. If testosterone concentration is less than 500 ng/dL, the dose is increased from 100 mg to 125 mg daily.

The dose of recombinant human growth hormone is about 5 μg/kg/day. In order to acclimate the patient to rhGH and reduce the potential for adverse events, the patient is to receive about 2.5 μg/kg/day, for about the first 28 days before increasing the dose to about 5 μg/kg/day.

### EXAMPLE 2

### Nutraceutical Composition Table for a Seven-Day Regimen

A seven-day regimen for administrating the nutraceutical composition is shown below in Table 1 in which all nutrients are offered each day.

TABLE 1

| Nutrient | Day 1 | Day 2 | Day 3 | Day 4 | Day 5 | Day 6 | Day 7 |
|---|---|---|---|---|---|---|---|
| Carnitine (mg) | 3,000 | 3,000 | 3,000 | 2,000 | 2,000 | 2,000 | 2,000 |
| Magnesium (mg) | 400 | 400 | 300 | 300 | 300 | 200 | 200 |
| Vitamin C (mg) | 500 | 500 | 400 | 400 | 300 | 200 | 200 |
| Selenium (μg) | 300 | 300 | 300 | 300 | 300 | 300 | 300 |
| Coenzyme Q10 (mg) | 300 | 300 | 300 | 200 | 200 | 200 | 200 |
| Alpha-lipoic acid (mg) | 100 | 100 | 100 | 100 | 100 | 100 | 100 |
| Tocopherol Mix (mg) | 100 | 100 | 200 | 400 | 400 | 400 | 400 |
| Cartenoid Mix (mg) | 100 | 100 | 200 | 400 | 400 | 400 | 400 |

### EXAMPLE 3

### Nutraceutical Composition Table for a Four-Week Regimen

A four-week regimen for administering the nutraceutical composition is shown below in Table 2 in which only carnitine, magnesium and selenium (water-soluble components) and coenzyme Q₁₀ are administered during the first week.

TABLE 2

| Nutrient | Week 1 | Week 2 | Week 3 | Week 4 |
|---|---|---|---|---|
| Carnitine (mg) | 3,000 | 2,000 | 2,000 | 1,500 |
| Magnesium (mg) | 100 | 200 | 150 | 100 |
| Vitamin C (mg) | — | 200 | 200 | 200 |
| Selenium (μg) | 200 | 200 | 200 | 200 |
| Coenzyme Q10 (mg) | 50 | 200 | 25 | 25 |
| Alpha-lipoic acid (mg) | — | 25 | 50 | 75 |
| Tocopherol Mix (mg) | — | 25 | 200 | 300 |
| Cartenoid Mix (mg) | — | 20 | 20 | 20 |

### EXAMPLE 4

### A Proposed In Vivo Experimental Protocol

The in vivo experimental protocol is devised to test the viability of utilizing testosterone, recombinant human growth hormone (rHGH), and a nutraceutical composition of the present invention for increasing whole body lean mass, skeletal mass, and muscle strength, while decreasing whole body fat mass to a greater extent than the combination of testosterone and growth hormone alone.

The in vivo experimental model employed comprises sexually mature, intact male rats that are 60 days old assigned to Group I, and sexually mature, hypophysectomized, castrated male rats that are 60 days randomly assigned to one of the following groups:

Group II: Hypophysectomized, castrated rats, saline treated daily;

Group III: Hypophysectomized, castrated rats treated daily with the combination of testosterone and growth hormone; and

Group IV: Hypophysectomized, castrated rats treated daily with the combination of testosterone, growth hormone and the nutraceutical compositions of the present invention.

Outcome measures:

The mitochondrial energy production (i.e., ATP production) in each of the groups is monitored by measuring the whole body and appendicular skeletal muscle mass. These measurements are made at baseline and after two weeks of treatment using Multi-Spectral Quantitative-Magnetic Resonance Imaging through a Siemens 3.0 Tesla MRI device. Rats are imaged at 3.0T with high resolution, mixed turbo spin echo pulse sequence before and after treatment. Directly-acquired images are Q-MRI post-processed, generating multi-spectral and whole-body Q-MRI data sets. The resulting data sets are used as input to a dual-space clustering segmentation algorithm targeting fat and muscle separately. Resulting whole-body fat segment are sub-segmented into subcutaneous, mesenteric, retroperitoneal, and epididymal components. Muscle spectral properties are studied by gaussian deconvolution.

The body weight of each animal is recorded on a daily basis. The wet weight of levator ani, quadriceps femoris, gastrocnemius, and soleus muscle groups are recorded. Serum testosterone, rHGH, and insulin-like growth factor 1 (IGF-1) levels are measured by radioimmunoassay. The whole body fat mass is measured by Multi-Spectral Quantitative-MRI. The wet weight of epididymal, retroperitoneal, mesenteric, and interscapular subcutaneous fat mass are recorded. The intramuscular mRNA, protein expressions of IGF-1, androgen receptor, and MyoD, myogenin, and major histocompatibility complex (MHC) are measured by reverse transcription and quantitative real time polymerase chain reaction (PCR).

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A method for rejuvenating cells having a lower energy production state to a higher energy production state in a warm-blooded animal including a human, said method comprising the steps of:
inducing the lower energy production state cells to increase mitochondrial energy production toward said higher energy production state; and
supplying a nutraceutical composition for uptake by the higher energy production state cells in amounts sufficient to satisfy the metabolic requirements resulting from the increase in mitochondrial energy production.

2. The method of claim 1 further comprising:
stimulating the lower energy production state cells to increase production of adenosine triphosphate, wherein the nutraceutical composition satisfies the metabolic requirements resulting from the increased production of adenosine triphosphate in the cells.

3. The method of claim 1 further comprising:
generating an induced energy nutrient deficient state in the mitochondria, wherein the nutraceutical composition satisfies the metabolic requirements of the induced energy nutrient deficient state in the mitochondria.

4. The method of claim 1 wherein the step of inducing the cells to increase mitochondrial energy production comprises administering a mitochondrial energy production-inducing agent to the cells.

5. The method of claim 1 further comprising:
measuring the progress of cell rejuvenation attained, and
adjusting the level of mitochondrial energy production for optimizing the cell rejuvenation progress in slowing or reversing the effects of ageing.

6. The method of claim 4 wherein the mitochondrial energy production-inducing agent is a hormone.

7. The method of claim 6 wherein the hormone is selected from the group consisting of growth hormone, a steroid, and mixtures thereof.

8. The method of claim 7 wherein the hormone is selected from the group consisting of recombinant human growth hormone (rhGH), testosterone, and mixtures thereof.

9. The method of claim 8 wherein an effective amount of the testosterone is administered to the patient to maintain a testosterone blood concentration of at least 500 ng/dL.

10. The method of claim 9 wherein the testosterone blood concentration is from about 500 ng/dL to 1000 ng/dL.

11. The method of claim 8 wherein the recombinant human growth hormone (rhGH) is administered at a dosage regimen of at least 2.5 μg/kg/day.

12. The method of claim 11 wherein the recombinant human growth hormone (rhGH) is administered at a dosage regimen of from about 2.5 μg/kg/day to 5 μg/kg/day.

13. The method of claim 1 wherein the nutraceutical composition comprises an energy nutrient capable of supplying the nutrients needed to meet and sustain the higher energy production state.

14. The method of claim 13 further comprising:
measuring the progress of cell rejuvenation attained, and
adjusting the formulation of the energy nutrients in the nutraceutical composition for optimizing the cell rejuvenation progress in slowing or reversing the effects of ageing.

15. The method of claim 13 wherein the energy nutrient is selected from the group consisting of a water-soluble energy nutrient, a fat-soluble energy nutrient, and mixtures thereof.

16. The method of claim 15 further comprising administering a dosage regimen, wherein an effective amount of each of said water-soluble energy nutrient and fat-soluble energy nutrient is maintained in the body of the warm-blooded animal including human.

17. The method of claim 16 wherein the water-soluble energy nutrient is selected from the group consisting of carnitine, magnesium, vitamin C, selenium and mixtures thereof,

18. The method of claim 15 wherein the fat-soluble energy nutrient is selected from the group consisting of tocopherols, carotenoids, coenzyme Q10, alpha-lipoic acid, and mixtures thereof.

19. The method of claim 15 further comprising varying over a period of time the effective dosage amounts of at least one of said water-soluble energy nutrient and fat-soluble energy nutrient for avoiding potentially adverse effects associated with excess stores of the fat-soluble energy nutrient in the body of the warm-blooded animal including human.

20. The method of claim 13 wherein the energy nutrient is selected from the group consisting of amino acids, quaternary amines, vitamins, dietary minerals, chelating agents, enzymes, ubiquinones, antioxidants, essential fatty acids, carotenoids, tocopherols, and mixtures thereof.

21. The method of claim 20 wherein the energy nutrient is selected from the group consisting of carnitine, magnesium, vitamin C, selenium, coenzyme Q10, α-lipoic acid, vitamin E, beta-carotene, and mixtures thereof.

22. The method of claim 1 wherein the nutraceutical composition comprises:
carnitine in an amount of from about 50 mg to 15,000 mg;
magnesium in an amount of from about 10 mg to 2,000 mg;
vitamin C in an amount of from about 10 mg to 1,000 mg;
selenium in an amount of from about 5 ug to 500 ug;
coenzyme Q10 in an amount of from about 5 mg to 500 mg;
α-lipoic acid in an amount of from about 5 mg to 600 mg;
at least one tocopherol in an amount of from about 5 mg to 800 mg; and
at least one carotenoid in an amount of from about 1 mg to 600 mg.

23. The method of claim 22 wherein the nutraceutical composition comprises:
carnitine in an amount of from about 100 mg to 10,000 mg;
magnesium in an amount of from about 50 mg to 1,000 mg;
vitamin C in an amount of from about 50 mg to 600 mg;
selenium in an amount of from about 10 ug to 400 ug;
coenzyme Q10 in an amount of from about 10 mg to 400 mg;
α-lipoic acid in an amount of from about 10 mg to 250 mg;
at least one tocopherol in an amount of from about 10 mg to 500 mg; and
at least one carotenoid in an amount of from about 3 mg to 500 mg.

24. A nutraceutical composition for administration to a patient having received a mitochondrial energy production-inducing agent to elevate the energy production for the mitochondria from a lower energy production state to a higher energy production state, wherein the existence of the higher energy production state results in a deficiency of energy nutrients necessary to sustain the higher energy production state, said composition comprising at least one energy nutrient in an amount effective for supplying the nutrients needed to sustain the higher energy production state, and a nutraceutically acceptable carrier.

25. The nutraceutical composition of claim 24 wherein the mitochondrial energy production-inducing agent is a hormone.

26. The nutraceutical composition of claim 25 wherein the hormone is selected from the group consisting of growth hormone, a steroid, and mixtures thereof.

27. The nutraceutical composition of claim 26 wherein the hormone is selected from the group consisting of recombinant human growth hormone (rhGH), testosterone, and mixtures thereof.

28. The nutraceutical composition of claim 27 wherein an effective amount of the testosterone is administered to the patient to maintain a testosterone blood concentration of at least 500 ng/dL.

29. The nutraceutical composition of claim 28 wherein the testosterone blood concentration is from about 500 ng/dL to 1000 ng/d L.

30. The nutraceutical composition of claim 27 wherein the recombinant human growth hormone (rhGH) is administered at a dosage regimen of at least 2.5 μg/kg/day.

31. The nutraceutical composition of claim 30 wherein the human growth hormone is administered at a dosage regimen of from about 2.5 μg/kg/day to 5 μg/kg/day.

32. The nutraceutical composition of claim 24 wherein the at least one energy nutrient is selected from the group consisting of a water-soluble energy nutrient, a fat-soluble energy nutrient, and mixtures thereof.

33. The nutraceutical composition of claim 32 wherein the water-soluble energy nutrient is selected from carnitine, magnesium, vitamin C, selenium and mixtures thereof.

34. The nutraceutical composition of claim 32 wherein the fat-soluble energy portion is selected from the group consisting of tocopherols, carotenoids, coenzyme Q10, alpha-lipoic acid, and mixtures thereof.

35. The nutraceutical composition of claim 32 comprising a dosage regimen wherein an effective amount of each of said water-soluble and fat-soluble energy nutrients is maintained in the body.

36. The nutraceutical composition of claim 35 wherein the dosage regimen comprises daily amounts of water-soluble energy nutrients and non-daily amounts of fat-soluble energy nutrients.

37. The nutraceutical composition of claim 32 wherein said at least one of said water-soluble energy nutrient and fat-soluble energy nutrient are present in effective dosage amounts for avoiding potentially adverse effects associated with excess stores of the fat-soluble energy nutrient in the body of the warm-blooded animal including human.

38. The nutraceutical composition of claim 24 wherein the energy nutrient is selected from the group consisting of amino acids, quaternary amines, vitamins, dietary minerals, chelating agents, enzymes, ubiquinones, antioxidants, essential fatty acids, carotenoids, tocopherols, and mixtures thereof.

39. The nutraceutical composition of claim 38 wherein the energy nutrient is selected from the group consisting of carnitine, magnesium, vitamin C, selenium, coenzyme Q10, α-lipoic acid, vitamin E, beta-carotene, and mixtures thereof.

40. The nutraceutical composition of claim 24 wherein the energy nutrient comprises:
carnitine in an amount of from about 50 mg to 15,000 mg;
magnesium in an amount of from about 10 mg to 2,000 mg;
vitamin C in an amount of from about 10 mg to 1,000 mg;
selenium in an amount of from about 5 ug to 500 ug;
coenzyme Q10 in an amount of from about 5 mg to 500 mg;
α-lipoic acid in an amount of from about 5 mg to 600 mg;
at least one tocopherol in an amount of from about 5 mg to 800 mg; and
at least one carotenoid in an amount of from about 1 mg to 600 mg.

41. The nutraceutical composition of claim 40 wherein the energy nutrient comprises:
carnitine in an amount of from about 100 mg to 10,000 mg;
magnesium in an amount of from about 50 mg to 1,000 mg;
vitamin C in an amount of from about 50 mg to 600 mg;
selenium in an amount of from about 10 ug to 400 ug;
coenzyme Q10 in an amount of from about 10 mg to 400 mg;
α-lipoic acid in an amount of from about 10 mg to 250 mg;
at least one tocopherol in an amount of from about 10 mg to 500 mg; and
at least one carotenoid in an amount of from about 3 mg to 500 mg.
